(19) United States
(12) Patent Application Publication
(10) Pub. No.: US 2024/0285226 A1
(43) Pub. Date: Aug. 29, 2024

(54) Automated Allergy Office

(71) Applicants: Ty L. Prince, Knoxville, TN (US); Herman J. Novak, Maryville, TN (US)

(72) Inventors: Ty L. Prince, Knoxville, TN (US); Herman J. Novak, Maryville, TN (US)

(21) Appl. No.: 18/657,012

(22) Filed: May 7, 2024

Related U.S. Application Data
(63) Continuation-in-part of application No. 18/185,827, filed on Mar. 17, 2023, now Pat. No. 11,998,359.
Application No. 18/185,827 is a continuation-in-part of application No. 18/078,856, filed on Dec. 9, 2022, now Pat. No. 11,911,172.
(60) Provisional application No. 63/362,623, filed on Apr. 7, 2022.
(60) Provisional application No. 63/289,350, filed on Dec. 14, 2021.

(51) Int. Cl. A61B 5/00; A61B 90/00; A61B 90/50; G16H 40/67

(52) U.S. Cl. CPC A61B 5/411; A61B 5/7264; G16H 40/67; A61B 2090/365; A61B 2090/502

(57) ABSTRACT

An automated allergy office system having an augmented reality headset for viewing a target location on a patient, where the target location is to be used for application of an allergen. The augmented reality headset includes artificial intelligence that at least one of visually identifies, grades, records, and displays a message summarizing information within the target location, including at least one of wheal color, wheal flare, skin color, skin discoloration, skin temperature, and location on the patient.

### PRIORITY

This application claims priority on, and is a continuation-in-part application of prior pending U.S. patent application Ser. No. 18/185,827 filed 2023 Mar. 17,
which claims benefit of Ser. No. 63,362,623 filed 2022 Apr. 7
and is a CIP of Ser. No. 18/078,856 filed 2022 Dec. 9,
which claims benefit of Ser. No. 63,289,350 2021 Dec. 14, the entirety of the disclosures of which are incorporated herein by reference.

### FIELD

The present invention relates to allergy testing, and more particularly to computer-based assessment of allergy skin results in a minimally invasive manner. The present invention also uses artificial intelligence and augmented reality to guide medical professionals to perform their testing and injection duties with significantly fewer mistakes.

### INTRODUCTION

To avoid an allergic reaction, people need to know what a patient is allergic to. Skin testing is one way a doctor can check on what causes your symptoms. These tests use extracts (a concentrated liquid form) of common allergens such as pollen, mold, dust mites, animal dander, and foods. Once those get under the skin of the patient, they could trigger a reaction called a wheal and flare. An allergy may trigger an itch, like a mosquito bite. That reaction is how the doctor can tell a patient's allergies. When a patient has an allergy, the patient's immune system will make antibodies and set off chemicals to fight off the trigger.

What is needed is an allergy testing system and method that tends to reduce false positives or negatives, improve the patient experience, reduce the cost of the current manual processes, or speed the billing process.

### SUMMARY

The above and other needs are met by an automated allergy office system having an augmented reality headset for viewing a target location on a patient, where the target location is to be used for application of an allergen. The augmented reality headset includes artificial intelligence that at least one of visually identifies, grades, records, and displays a message summarizing information within the target location, including at least one of wheal color, wheal flare, skin color, skin discoloration, skin temperature, and location on the patient.

In various embodiments according to this aspect of the disclosure, the augmented reality headset further records an image of the target location during at least one of prior to the application of the allergen, at the application of the allergen, and after the application of the allergen. In some embodiments, if the target location has a skin discoloration as identified by the artificial intelligence, the artificial intelligence identifies a different target location to avoid the skin discoloration. In some embodiments, if the target location has a skin discoloration as identified by the artificial intelligence, the artificial intelligence advises a user to relocate the target location to avoid the skin discoloration. In some embodiments, the augmented reality headset enables an objective reading of at least one of the wheal and the flare from at least one of a tattoo and a darker complexion on the patient. In some embodiments, the augmented reality headset prompts a user through at least one of an allergy testing process and an immunology injection process by communicating process steps to at least one of a desktop computer, a tablet, a smart phone, and the augmented reality headset. In some embodiments, the artificial intelligence enables portions of the allergy testing process to be performed remotely from the from the augmented reality headset.

### BRIEF DESCRIPTION OF THE DRAWINGS

Further advantages of the embodiments are apparent by reference to the detailed description when considered in conjunction with the figures, which are not to scale so as to show the details more clearly, wherein like reference numbers indicate like elements throughout the several views, and wherein:

FIG. 1 depicts a first embodiment of the automated allergy office of the present disclosure.

FIGS. 2A, 2B, 2C, 2D, and 2E depict the operations performed in the simplified schematic layout of the automated allergy office of the present disclosure.

### DESCRIPTION

A first embodiment of the automated allergy office system and method of the present disclosure takes a medical professional through an allergy testing process and a follow-on immunology injection process step-by-step using LED computer screen tables, computer tablets, smart phones, an internal office computer system accessing the Patient's medical records, and an augmented reality headset. The augmented reality headset technology and artificial intelligence are layered onto an improved manual system to minimize false positives and negatives, to improve the patient experience, resulting in cost savings for the doctor and patient and enabling portions of the allergy testing to be performed remotely from the office of the allergist, either by the patient or by a trained medical technician. The testing vials, the patient specific immunology vials, and the skin test target tags and the patient, in some embodiments, each deploy either radio frequency ID tags, Bluetooth low energy tags, or near field communication tags.

A second embodiment of the automated allergy office system of the present disclosure comprises back or arm target stickers used by artificial intelligence and augmented reality headset as sites such that the system determines target locations of where to perform scratch or injection points, wherein moles, skin tags, pre-existing rash or tattoo, that may yield a false positive or negative display a message for a medical technician to move the back or arm target sticker to a scratch or injection point away from the mole, skin tag, pre-existing rash or tattoo. If the patient has the mole or skin tag as identified by the artificial intelligence or the augmented reality headset, a second test site is identified. The second test site reduces the likelihood of false positives and negatives. The automated allergy office system of the present disclosure may be initiated either in the office of the allergist or remotely. The automated allergy office system of the present disclosure may be periodically updated either in the office of the allergist or remotely.

A third embodiment of the automated allergy office system of the present disclosure including an augmented reality headset that projects target allergen injection points on the image of the back or arm of the patient relative to the target location wherein if the augmented reality headset detect a mole or skin tag at the target location is moved to avoid the mole or skin tag. The augmented reality headset combined with voice recognition technology interprets spoken commands of a wearer of the augmented reality headset. The augmented reality headset reads and records the wheal color, and diameter, and the diameter of the flare. In another embodiment, the augmented reality headset also reads and records an infrared signature of a wheal reaction site and flare reaction site. A second embodiment of the automated allergy office system of the present disclosure also includes means for retesting negative test results. The retesting of the negative test results is performed using prefilled vials and syringes, wherein the augmented reality headset monitors the content of each vial, each vial location in a testing tray, temperature of the vials to ensure they do not get too hot or too cold and the location of each skin test. In addition, the augmented reality headset monitors injection sites and the location of the used skin testers and the location of used vials.

A fourth embodiment of the automated allergy office system of the present disclosure comprises artificial intelligence learning of the correct interpretation of wheal, flare, color, and infrared temperature measurement, including grading, the artificial intelligence through an augmented reality headset, recording each test site to ensure that the interpretation of the injection site is analyzed correctly. The augmented reality headset captures an image of a test site immediately after an allergen is administered. The augmented reality headset also captures an image of a test site at about a 20-minute interval after the tests are completed. The automated allergy office system of the present disclosure further comprise means for updating artificial intelligence models with current values. Such updated artificial intelligence models are designed for achieving optimal values while safeguarding patient identity in an artificial intelligence library.

The automated allergy office of the present disclosure comprises preloaded allergy testing vials, preloaded allergy multi testing trays and an automated process for filling patient immunology vials. It also uses the latest technology for patient data entry (phone or tablet app), identifying a patient (a biometric-such as face, finger, voice, or a combination of thereof), processing the patient when the patient visits the office, the ability for home allergy testing, an augmented reality system to help to eliminate mistakes in the testing process, inventory, the data recording process, the analysis process, the immunology process and the billing process. This automated allergy office of the present disclosure eliminates the need to place marks on a patient's back or arm, to identify allergen test sites, it also eliminates the need to put marks on a patients arm or back for subsequent intradermal injection tests performed only if individual tests are negative on percutaneous tests. Since the system has a built-in timer, the nurse will be notified when a user reaches the 20-minute time has elapsed when the scratches or injections were initially applied. This time out period will instruct the nurse to return to the exam room where the positive, negative analysis will be completed. The system is designed to step the nurse or doctor through the allergy testing process and the follow-on immunology injection process with the use of LED computer screen tables, computer tablets, smart phones, an internal office computer system (patient records and billing) and augmenter reality headset.

The automated allergy office system and method of the present disclosure reads wheal size change, flare size change, wheal, and flare color as well as wheal and flare temperature. Using the traditional method of reading wheal size change and flare size and color, it is difficult to analyze these changes on a person with dark skin or tattoos. By adding wheal and flare color analysis and wheal and flare temperature change, the interpretation of a positive or negative reaction on a patient with a darker skin complexion or with tattoos will be greatly improved.

Still other aspects of the processes for utilizing the automated allergy office of the present disclosure will become apparent to those skilled in this art from the following description wherein there is shown and described various embodiments, simply by the way of illustration of the modes contemplated for carrying out the present disclosure. As will be realized, the present disclosure is capable of different embodiments, and its several details are capable of modification in various, obvious aspects all without departing from the disclosure. Accordingly, the drawings and descriptions will be regarded as illustrative in nature and not as restrictive.

With reference now to the drawings, there are depicted all of the claimed elements of the various embodiments, although all claimed embodiments might not be depicted in a single drawing. Thus, it is appreciated that not all embodiments include all of the elements as depicted, and that some embodiments include different combinations of the depicted elements. It is further appreciated that the various elements can all have many different configurations, and are not limited to just the configuration of a given element as depicted. As indicated above, the elements of the drawings as depicted are not to scale, even with respect one to another, and relative size or thickness of one element cannot be determined by the aspect ratios of that element or with reference to any dimension of another element.

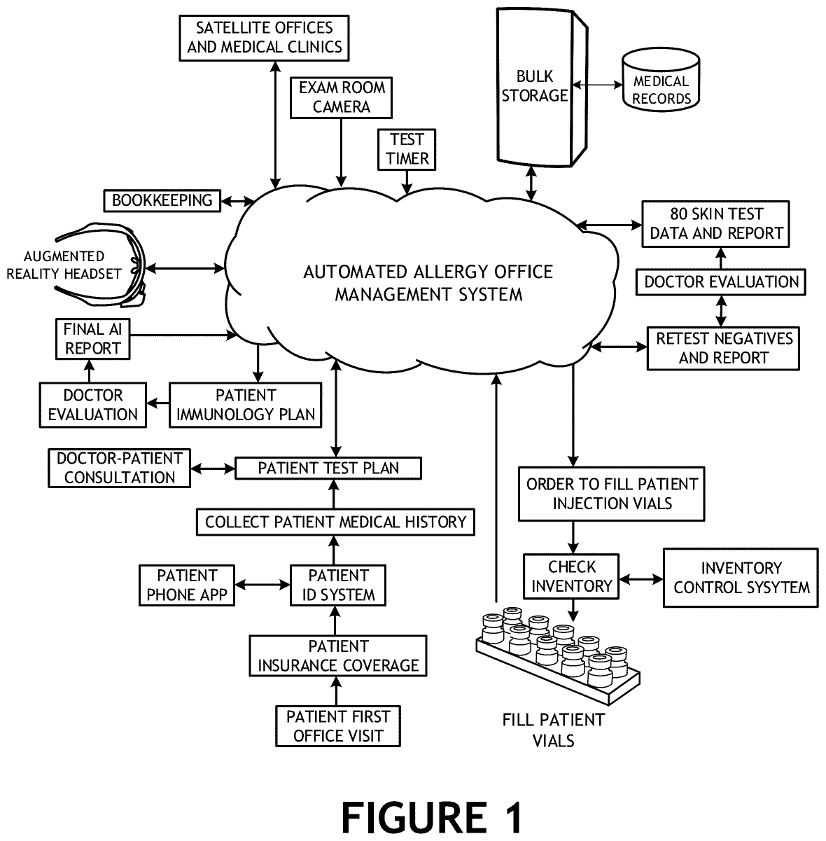

Referring now to the drawings, FIG. 1 depicts a first embodiment of the automated allergy office of the present disclosure.

A phone or tablet app for the patient is used to input personal and medical information prior to or during the initial office visit. This includes symptoms, photographs of skin rashes with the color of rash and temperature of any rash being recorded as part of the patient's record along with any other indications that are unusual or new.

The patient schedules a consultation with the allergist by phone or tablet app. The consultation can either be a visit to the allergist's office, a visit to some other medical facility, or a phone consultation. If the initial visit between the patient and doctor is done remotely, for example, if the patient is home-bound, the skin tests can be performed by a nurse or medical technician from the doctor's office visiting the patient. Similarly, if the patient is home-bound and follow-up testing is required, the tests can be performed by a nurse or medical technician from the doctor's office visiting the patient. Satellite offices and medical clinics can also be incorporated into the system.

The patient's medical history and insurance information is obtained during the initial doctor-patient contact. The medical history may also be submitted by written forms, by phone, or a tablet app or entered by the patient on a website. This includes symptoms, photographs of skin rashes with the color of rash and temperature of any rash being recorded as part of the patient's record along with any other indications that are unusual or new.

Also, one or more of the patient's biometric identifiers are obtained (e.g.—face, finger, voice). Bimodal identification is used in one embodiment.

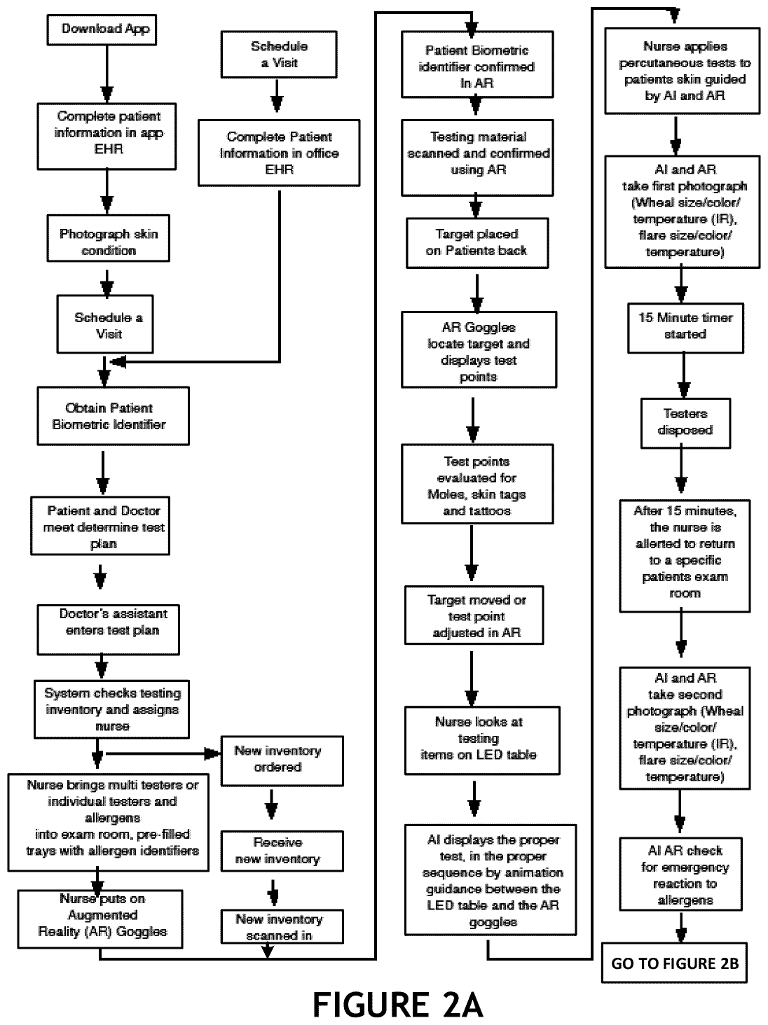

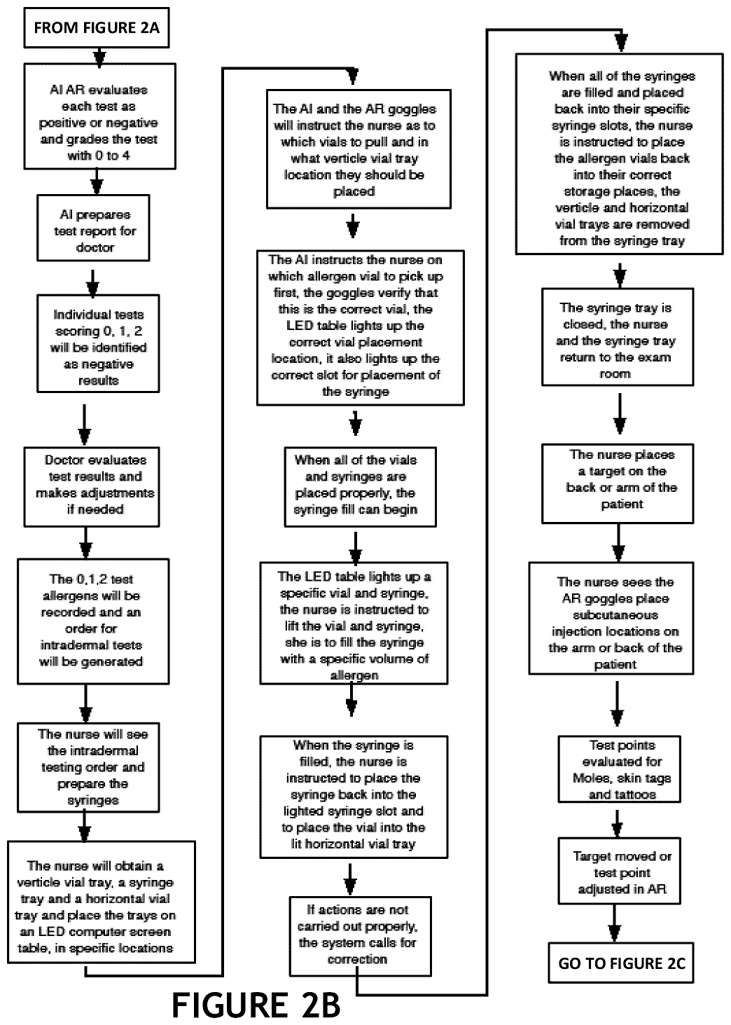

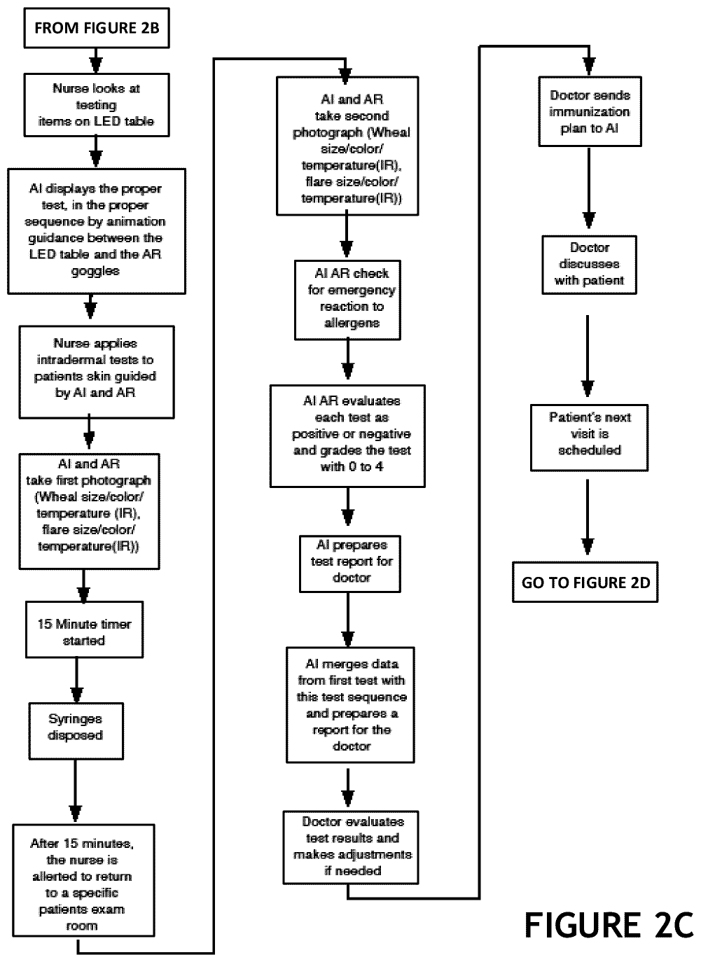

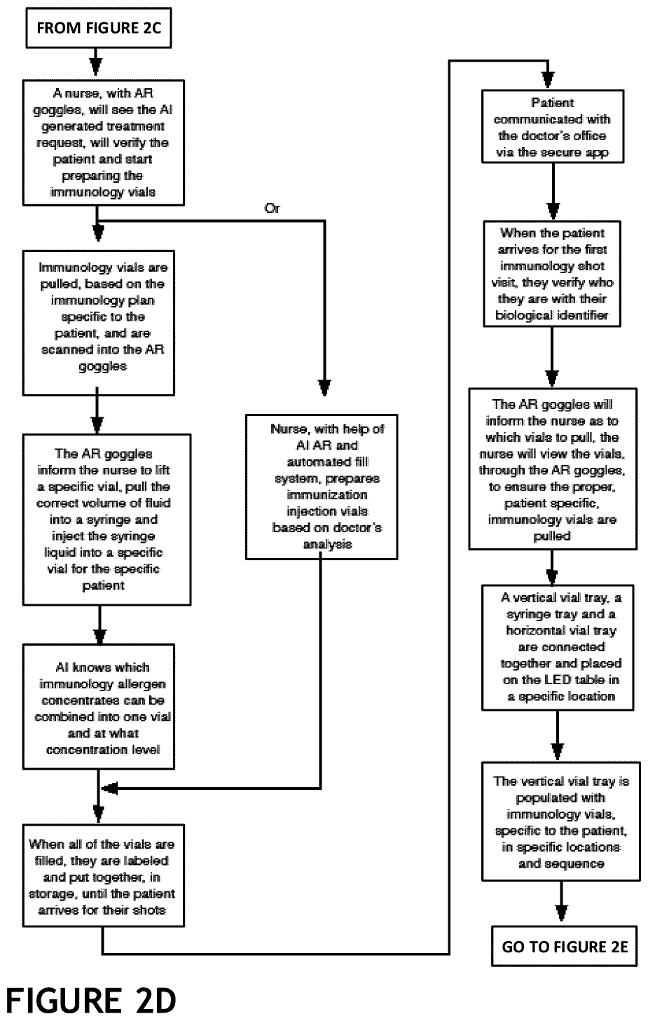

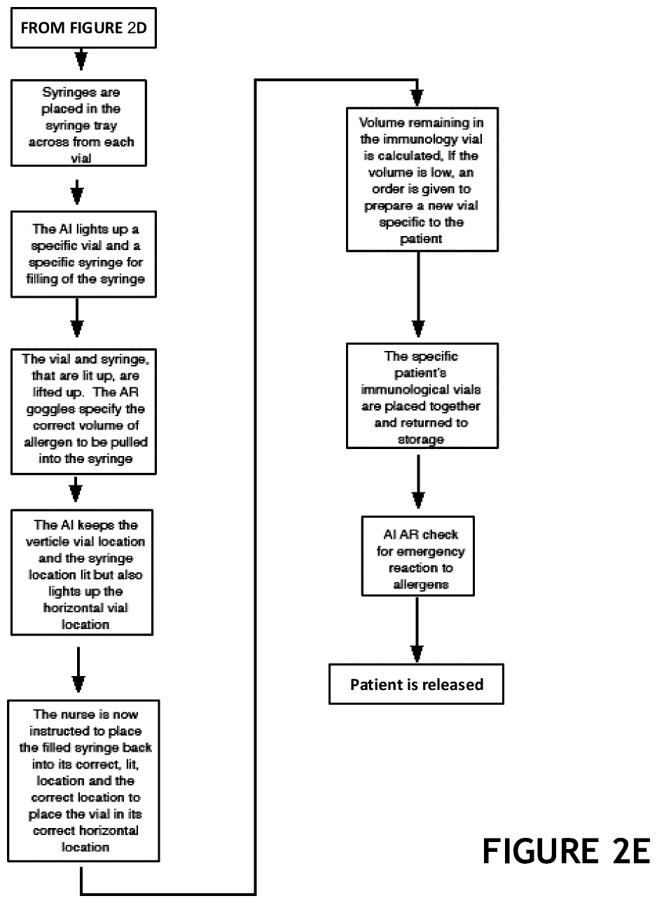

In addition, FIGS. 2A, 2B, 2C, 2D, and 2E depict the operations performed in a simplified schematic layout of the automated allergy office of the present disclosure.

The automated allergy office of the present disclosure includes clear, single tester, multi tester and vial trays. The testing vials, patient specific immunology vials, and skin test target tags, will use the latest ID tag technology.

RFID tags which have an antenna and receives power, via an RF field, generated by the RFID tag reader are used. Bluetooth low energy tags are also used. Near field communication tags are also used. Wiliot provides near field communication tags for the electronic tracking of medication, flexible electronic connectivity, and battery-free sensor tags. These tags enable the electronic tracking and monitoring of medication dispensing and intake. The kit is a multi-vial box covered with a cardboard layer of continuously numbered, perforated areas corresponding to the individual compartments. When the user opens a perforation at the starter tab the removal of the vial will be tracked by means of integrated, printed electronics.

The Bluetooth low energy tags and the near field communication tags include electronics and memory imbedded in a tag that needs no external power source, it obtains its needed power by harvesting energy from the RF signals that are present in homes and offices, such as Wi-Fi router signals.

A near field communication tag supplied by Wiliot that is glued to a simple antenna printed on plastic or paper can authenticate the proximity of a product by transmitting an encrypted serial number along with weight and temperature data from a device the size of a postage stamp. Eliminating most of the components associated with traditional Bluetooth, these tags lower sale and maintenance costs to previously unachievable levels.

These tags communicate with the office automation system through secure Wi-Fi or even through the augmented reality headset.

This embodiment of the automated allergy office includes a table-top LED display.

The augmented reality headset, in some embodiments, includes voice recognition that understands the voice of the nurse, will read the color and size of the wheal and flare, and will read the infrared signature of the wheal and flare reaction site.

The driver for the augmented reality headset may be a computer, a tablet, or a secure Wi-Fi network.

The prefilled single tester tray, in some embodiments, uses a barcode, a QR-type code or some other identifier. The prefilled multi tester tray uses a barcode, a QR-type code, or some other identifier. Prefilled allergen vials use a barcode, a QR-type code, or some other identifier.

The trays are scanned with the augmented reality headset. Even though the trays are preloaded, the augmented reality headset serves to record what was given to the patient.

Back or arm target stickers are used by the artificial intelligence and an augmented reality headset as a location point such that the system knows were to display the scratch or injection points (moles, skin tags, pre-existing rash or tattoos, that could cause the system to locate a scratch point or an injection point that may give a false positive or negative, will display a message for the nurse to move the target sticker or will automatically move the injection point away from the mole, skin tag, pre-existing rash or tattoo).

The augmented reality headset captures the image of the test sites immediately after each test is completed as well as 20 minutes after the tests are completed.

The system guides the nurse through the testing and injection process. However, if for some reason the nurse exits the system or allows the system to go into a sleep mode, such as if there is something that interrupts the process and causes her to leave, when she returns, the system identifies the point in the process at which she stopped, and prompts her to continue from that point. Thus, she does not need to be concerned about either duplicating or skipping any of the procedures.

After twenty minutes, the nurse returns to the exam room so that the system can perform the final data collection and analysis. It is important to return to the exam room after the 20 minutes because some wheal and flare reactions can dissipate quickly after 25-30 minutes. An accurate analysis timed after 20 minutes will help to minimize the number of false positives or negatives.

If the artificial intelligence sees a reaction that may harm the patient, the system signals the nurse or doctor to see the patient immediately. The testing process is timed.

The automatic filling system for immunotherapy vials is identified and locked by the biometric identifier of the patient.

A first embodiment of the automated allergy office system and method of the present disclosure takes a medical professional through an allergy testing process and a follow-on immunology injection process step-by-step using LED computer screen tables, computer tablets, smart phones, an internal office computer system accessing the patient's medical records, and an augmenter reality headset. The augmented reality headset technology and artificial intelligence are layered onto an improved manual system to minimize false positives and negatives, to improve the patient experience patient, resulting in cost savings for the doctor and patient and enabling portions of the allergy testing to be performed remotely from the office of the allergist, either by the patient or by a trained medical technician. The testing vials, the patient specific immunology vials, and the skin test target tags, will, in some embodiments, each deploy either radio frequency ID tags, Bluetooth low energy tags, or near field communication tags.

A second embodiment of the automated allergy office system of the present disclosure comprises back or arm target stickers used by artificial intelligence and augmented reality headset as sites such that the system determines target locations of where to perform scratch or injection points, wherein moles, skin tags, pre-existing rash, or tattoo, that may yield a false positive or negative display a message for a medical technician to move the back or arm target sticker to a scratch or injection point away from the mole, skin tags, pre-existing rash, or tattoo. If the patient has the mole, skin tags, pre-existing rash, or tattoo, as identified by the artificial intelligence or the augmented reality headset, a second test site is identified. The second test site reduces the likelihood of false positives and negatives. The automated allergy office system of the present disclosure may be initiated either in the office of the allergist or remotely. The automated allergy office system of the present disclosure may be periodically updated either in the office of the allergist or remotely.

A third embodiment of the automated allergy office system of the present disclosure including an augmented reality headset that projects target allergen injection points on the image of the back or arm of the patient relative to the target location wherein if the augmented reality headset detect a mole, skin tag, pre-existing rash, or tattoo, at the target location is moved to avoid the mole, skin tag, pre-existing rash, or tattoo. The augmented reality headset combined with voice recognition technology interprets spoken commands of a wearer of the augmented reality headset. The augmented reality headset reads and records the wheal color, wheal size and change in color as well as flare size. In another embodiment, the augmented reality headset also reads and records an infrared signature of a wheal reaction site and flare reaction site. A second embodiment of the automated allergy office system of the present disclosure also includes means for retesting negative test results. The retesting of the negative test results is performed using prefilled vials and syringes, wherein the augmented reality headset monitors the content of each vial, each vial location in a testing tray and the location of each skin tester. In addition, the augmented reality headset monitors injection sites and the location of the used skin testers and the location of used vials.

A fourth embodiment of the automated allergy office system of the present disclosure comprises artificial intelligence learning of the correct interpretation of wheal, flare, color, and infrared temperature measurement, including grading, the artificial intelligence through an augmented reality headset, recording each test site to ensure that a negative reaction does not occur to the injection. The augmented reality headset captures an image of a test site immediately after an allergen is administered. The augmented reality headset also captures an image of a test site at about a 15-minute interval after the tests are completed. The automated allergy office system of the present disclosure further comprise means for updating artificial intelligence models with current values. Such updated artificial intelligence models are designed for achieving optimal values while safeguarding patient identity in an artificial intelligence library.

Still other aspects of the processes for utilizing the automated allergy office of the present disclosure will become apparent to those skilled in this art from the following description wherein there is shown and described in the embodiments of this disclosure, simply by the way of illustration of the best modes contemplated for carrying out the present disclosure. As will be realized, the present disclosure is capable of different embodiments, and its several details are capable of modification in various, obvious aspects all without departing from the invention. Accordingly, the drawings and descriptions will be regarded as illustrative in nature and not as restrictive.

The database and artificial intelligence for reaction interpretation examines the size change of the wheal, the size change of the flare, any color change of the wheal and flare, and infrared measurement of any temperature change.

The artificial intelligence analysis is comprehensive. A printout of artificial intelligence analysis of wheals, flares, color, temperature, and grade. Paper, tablet, or computer accessible artificial intelligence analysis are provided for the doctor to evaluate and adjust as needed. Negative test results from the percutaneous scratch testing are retested with prefilled vials and syringes (the augmented reality headset read the content of each vial, their location in the testing trays as well as the location of each syringe, the augmented reality headset monitor the injection site as well as the location of the used syringes and the location of the used vials).

The verified negatives are recorded and merged with the initial percutaneous allergy test results. If a negative retest shows a positive from the intradermal injection retest, the data is merged with the initial test data for the doctor's analysis and conclusion.

After the doctor adjusts the artificial intelligence analysis, the data is entered into the patient's record and is used for the automatic preparation of the patient's immunological shot vials.

A copy of this record, with patient-specific data removed, is put into the artificial intelligence learning database, this will improve the artificial intelligence results in the future. A system of concentrated allergens, some being grouped together, are driven by the doctor's immunological plan with specific volumes pumped into the vials that will be used for the patient immunological shots. The system generates tags for each vial with the patient's name, contents, coded biometric data, expiration date, manufacturer, lot number of stock bottle, expiration date of stock bottle, stock source from manufacturer, vial temperature and the vial number (e.g.—1 of 4).

For examples of how artificial intelligence is used in the automated office of the present disclosure, see United States publication 20200202245 (Lesnik; et al.) entitled “A Probabilistic Data Classifier System and Method Thereof” filed Oct. 14, 2018, and United States publication 20190065464 (Finley; et al.) entitled “Artificial Intelligence Scribe” filed Mar. 8, 2018.

Before each shot is given to a patient, the unique patient identifier is checked to confirm patient identity, the patient's shot vials are scanned, and the injection volume is displayed for the nurse or medical technician. After the shot is given, the shot vials are scanned again. Finally, the artificial intelligence, through the augmented reality headset records each shot site to ensure that the patient does not experience a negative reaction to the injection. If the patient complains of a negative reaction, or if the augmented reality headset detects a negative reaction, an alert is given to the nurse, medical technician, and the doctor requesting immediate help. The time to administer the immunology shots is also recorded.

As the doctor, nurse, and medical personnel become familiar with the images and animation displayed in the augmented reality headset, he or she can choose to speed up or eliminate portions of the animation. However, the sped-up visuals will continue to display all the key elements of the display sequence to ensure that the proper process and procedure is followed.

As used herein, the term “augmented reality headset” refers to a relatively new technology that integrates information from the real world and the virtual world “seamlessly”. Specifically, by means of techniques such as electronic computer processing, augmented reality technology applies virtual information to the real world by simulation and then superimposition, and the result is perceived by human senses. Therefore, a sensory experience that transcends reality is achieved. In this case, the real environment and the virtual object are superimposed in the same picture or space in real time. The augmented reality technology not only displays real-world information, but also displays virtual information at the same time, so that the two kinds of information complement and superimpose each other. In the visual augmented reality technology, the real world and computer graphics are multi-synthesized by using a helmet display. With maturity of the market, a variety of augmented reality products have emerged, especially head-mounted augmented reality headset. See, for example, United States publication 20200211420 (Buras; et al.) entitled “System and Method for Three-Dimensional Augmented Reality Guidance for Use of Medical Equipment” filed Dec. 26, 2019, and U.S. Pat. No. 10,197,803 (Badiali; et al.) entitled “Augmented Reality Glasses for Medical Applications and Corresponding Augmented Reality System” filed Mar. 27, 2015.

The automated allergy office of the present disclosure will result in portions of the allergy diagnosis and treatment moving out of the allergist's office to intermediate medical sites and to a certain degree of home testing, analysis, and treatment of allergies.

Still other aspects of the telehealth system of the present disclosure will become apparent to those skilled in this art from the following description wherein there is shown and described in the embodiments of this disclosure, simply by the way of illustration of the best modes contemplated for carrying out the present disclosure. As will be realized, the present disclosure is capable of different embodiments, and its several details are capable of modification in various, obvious aspects all without departing from the disclosure. Accordingly, the drawings and descriptions will be regarded as illustrative in nature and not as restrictive.

Throughout this application, various provisional applications, patents applications, PCT applications, and patents are referenced. The disclosures of these documents in their entireties are hereby incorporated by reference into this specification to more fully describe the state of the art to which this disclosure pertains.

As used herein, the phrase “at least one of A, B, and C” means all possible combinations of none or multiple instances of each of A, B, and C, but at least one A, or one B, or one C. For example, and without limitation: A×1, A×2+B×1, C×2, A×1+B×1+C×1, A×7+B×12+C×113. It does not mean A×0+B×0+C×0.

The foregoing description of embodiments for this disclosure has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An automated allergy office system comprising an augmented reality headset for viewing a target location on a patient, where the target location is to be used for application of an allergen, the augmented reality headset including artificial intelligence that at least one of visually identifies, grades, records, and displays a message summarizing information within the target location, including at least one of a wheal color, a wheal flare, a skin color, a skin discoloration, a skin temperature, and a location on the patient.

2. The automated allergy office system of claim 1, wherein the augmented reality headset further records an image of the target location during at least one of prior to the application of the allergen, during the application of the allergen, and after the application of the allergen.

3. The automated allergy office system of claim 1, wherein if the target location has the skin discoloration as identified by the artificial intelligence, the artificial intelligence identifies a different target location to avoid the skin discoloration.

4. The automated allergy office system of claim 1, wherein if the target location has the skin discoloration as identified by the artificial intelligence, the artificial intelligence advises a user to relocate the target location to avoid the skin discoloration.

5. The automated allergy office system of claim 1, wherein the augmented reality headset enables an objective reading of at least one of the wheal color and the wheal flare from at least one of a tattoo and a darker complexion on the patient.

6. The automated allergy office system of claim 1, wherein the augmented reality headset prompts a user through at least one of an allergy testing process and an immunology injection process by communicating process steps to at least one of a desktop computer, a tablet, a smart phone, and the augmented reality headset.

7. The automated allergy office system of claim 6. wherein the artificial intelligence enables portions of the allergy testing process to be performed remotely from the augmented reality headset.
